(19) United States
(12) Patent Application Publication
(10) Pub. No.: US 2013/0261661 A1
(43) Pub. Date: Oct. 3, 2013

(54) SURGICAL STAPLER

(71) Applicant: Hadi A. Piraka, Northville, MI (US)

(72) Inventor: Hadi A. Piraka, Northville, MI (US)

(21) Appl. No.: 13/261,593

(22) PCT Filed: Aug. 5, 2011
(86) PCT No.: PCT/US11/46679

§ 371 Date: Jun. 17, 2013

Related U.S. Application Data
(60) Provisional application No. 61/370,985, filed on Aug. 5, 2010.

(51) Int. Cl. A61B 17/068; A61B 17/10

(52) U.S. Cl. CPC A61B 17/068; A61B 17/105
USPC 606/219; 227/176.1; 227/175.2; 227/180.1

(57) ABSTRACT

A stapling and cutting system for use by a physician in the cutting and stapling together of layers of body tissue. The system includes first and second handles that are pivotally coupled together at a pivot joint located between proximate and distal ends of each of handles. First and second jaws are coupled together and movable between an open and closed position. Mounted on a distal end of the first jaw for positioning on one side of the layers of tissue is a disposable staple cartridge, which includes a plurality of staples. An anvil, mounted on the distal end of the second jaw, is positioned on opposing side of the tissue to be stapled. To manipulate the jaws, a closure mechanism couples the first and second handles to the first and second jaws. The closure mechanism maintains the anvil and the staple cartridge substantially parallel to and opposite from one another when the first and second jaws are in the open position and moved to the closed position. When operated, the staples are discharged from the system and clinched when the first and second jaws are moved into the closed position.

### BACKGROUND

1. Field of the Invention

The present invention generally relates to surgical instruments and devices. More particularly, the present relates to a surgical cutting and stapling system for use in the stapling and cutting layers of a material together (e.g., the stapling and cutting of tissue layers during a surgical procedure).

2. Description of the Known Technology

A variety of devices have been previously proposed for dispensing staples used in joining together of body tissues.

One such device includes a pair of handles pivotably connected between a pair of end portions so as to open a pair of jaws when the handles are moved apart and close the jaws when the handles are moved together. A detent is used to aid the surgeon in properly clamping the layers of tissue before the staples are discharged and clinched through the tissues. The jaws of this system pivot relative to one another during closing such that the portion of the staples closest to the handles engage the opposite jaw prior to those staples located the farthest from the handles. By increasing the length of the jaws, this disparity can be reduced, but not eliminated. If the length of the jaws is shortened, however, this disparity increases.

### SUMMARY

In overcoming the above drawbacks and limitations, the present invention provides a stapling and cutting system for use by a physician in the cutting and stapling together of layers of body tissue. The stapling and cutting system includes a first handle and a second handle, the first and second handles being pivotally coupled together at a pivot joint located between proximate and distal ends of each of the first and second handles. A first jaw and a second jaw are coupled together and movable between an open position and a closed position. A disposable staple cartridge is mounted on a distal end of the first jaw for positioning on one side of the layers of tissue; the staple cartridge includes a plurality of staples. An anvil, mounted on the distal end of the second jaw, positioned on opposing side of the layers of tissue. To manipulate the jaws, a closure mechanism couples the first and second handles to the first and second jaws and causes movement of the first and second jaws from the open position to the closed position upon manipulation of the first and second handles. The closure mechanism positions the anvil and the staple cartridge substantially parallel to and opposite from one another when the first and second jaws are in the open position and also positions the anvil and the staple cartridge substantially parallel to and opposite from one another when the first and second jaws are in the closed position. When operated, the staples are discharged from the system and clinched when the first and second jaws are moved from the open position into the closed position.

In another aspect, the present invention provides a method for discharging a staple from a stapling and cutting system. The method comprising the steps of
providing a stapling and cutting system, the stapling and cutting system having a first jaw and a second jaw; the jaws being movable between open and closed positions and being operable by manipulation of a first handle and a second handle, a disposable staple cartridge located on the first jaw and including multiple staples, an anvil located on the second jaw generally opposite from the staple cartridge, a closure mechanism that couples the first and second handle to the first and second jaws;
moving the first and second handles toward each other under a first pressure to move the jaws from the open position toward the closed position while maintaining the staple cartridge and the anvil generally parallel to each other;
while keeping the staple cartridge and anvil substantially parallel to each other, moving the first and second handles toward each other under a second pressure and causing the jaws to move into the closed position, the second pressure being greater than the first pressure;
squeezing the handles at the second pressure whereby at least one staple is discharged from staple cartridge and clinched by interaction with the anvil; and
opening the jaws to permit removal discarding of the staple cartridge from the system.

Additional features and advantages of this invention will become readily apparent to persons skilled in the art after a review of the following description, with reference to the drawings and claims that are appended to and form a part of this specification

### BRIEF DESCRIPTION OF THE DRAWINGS

The drawings described herein are for illustration purposes only and are not intended to limit the scope of the present disclosure in any way.

FIG. 1 is a perspective view of a stapling and cutting system constructed according to the teachings of the present disclosure;

FIGS. 2A-2E are cross-sectional views of the anvil and disposable cartridge portions of the surgical stapling and cutting system of FIG. 1 according to other aspects of the present disclosure;

FIGS. 3A-3B represents an exploded perspective view of the disposable cartridge of FIG. 2.

FIGS. 4A-4C are schematic representations of a staple firing prevention mechanism shown before firing and after firing the staples in the surgical stapling and cutting system of FIG. 1, according to another aspect of the present invention;

FIG. 5 is a perspective view of an angled blade for use with the surgical stapling and cutting system of FIG. 1, according to another aspect of the present invention; and

FIG. 6 is a schematic representation of a method for using the surgical stapling and cutting system of FIG. 1, according to another aspect of the present invention.

### DETAILED DESCRIPTION

The following description is merely exemplary in nature and is in no way intended to limit the present disclosure or its application or uses. For example, in order to more fully illustrate the present system and its method of use, the stapling and cutting system is described throughout the present application in conjunction with a surgical procedure used to join layers of body tissue using staples. The incorporation and use of such a stapling and cutting system in conjunction with layers of cloth, fabric, plastic, and films, among others, is contemplated to be within the scope of the disclosure. In addition, the system can also be used to apply surgical clips or other fasteners when desired.

It should also be understood that throughout the present description and drawings, corresponding reference numerals indicate like or corresponding parts and features. In addition, the term “proximal” refers to a portion of the stapling and cutting system that is closer to the operator, while the term “distal” refers to a portion of the system that is further away from the operator.

The present disclosure generally provides a stapling and cutting system used to cut and join together layers of a material, such as body tissue, through the use of multiple staples. The stapling and cutting system generally has an open position in which an operator, such as a physician, can maneuver the system into place, and a closed position in which the staples are forced to join the layers of material together.

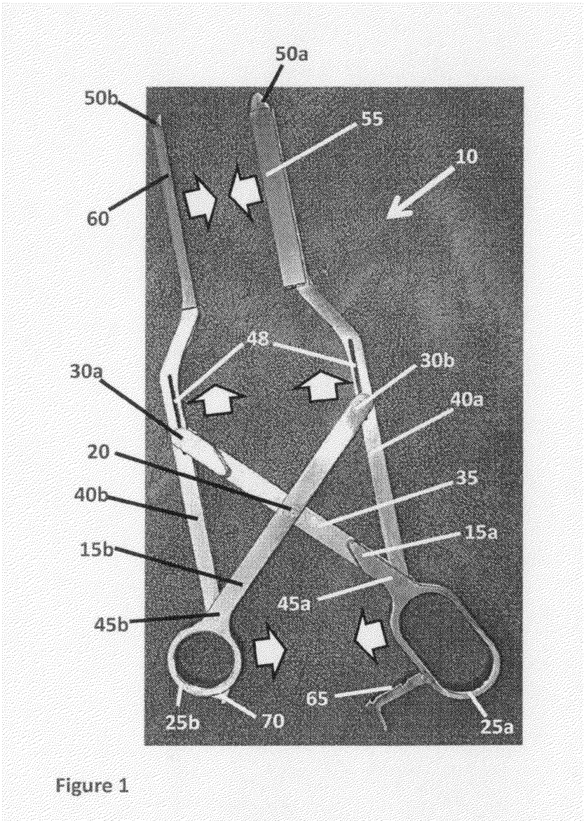

Referring to FIG. 1, the relationship between the various components of the stapling and cutting system 10 used to join layers of body tissue or other materials is shown to include a first handle 15a pivotally connected to a second handle 15b through a pivot joint/point 20 located between the proximal end 25a, 25b and distal end 30a, 30b of each handle 15a, 15b, respectively. The proximal end 25a of the first handle 15a is connected to the proximal end 45a of a first jaw 40a. Similarly, the proximal end 25b of the second handle 15b is connected to the proximal end 45b of a second jaw 40b. The distal end 30a of the first handle 15a is slidably coupled to the second jaw 40b through an elongated slot 48 formed in the second jaw 40b. Likewise, the distal end 30b of the second handle 15b is slidably coupled to the first jaw 40a through a similar elongated slot 48 in the first jaw 40a. The elongated slots 48 in the first and second jaws 40a, 40b are positioned lengthwise with the length of each jaw 40a, 40b. Preferably, the distal end 30a, 30b of each handle 15a, 15b has a pin or similar projection arising from the surface of the handle 15a, 15b that is slidably received by the elongated slot 48 in the corresponding jaw 40a, 40b. The interaction and movement of the pin or projection arising from the surface of the handles with respect to the slots 48 in the jaws 40a, 40b make up a key component of the closure mechanism of the associated stapling and cutting system 10.

The stapling and cutting system 10 is movable between an open position and a closed position. Still referring to FIG. 1, in the open position each handle 15a, 15b is positioned such that the proximal end 25a, 25b of each handle 15a, 15b is separated from one another, which causes the distal end 30a, 30b of each handle 15a, 15b to couple with the jaws 40a, 40b at one end (e.g., first end) of the corresponding slot 48 located in the jaw 40a, 40b. When the stapling and cutting system 10 is moved to its closed position, the proximal end 25a, 25b of each handle 15a, 15b is moved towards one another, thereby, forcing the distal end 30a, 30b of each handle 15a, 15b to slide towards the opposite end (e.g., second end) of the corresponding slot 48. This sliding motion in turn causes the distal end 50a, 50b of the jaws 40a, 40b to move towards one another. Thus, when the proximal end 25a, 25b of the handles 15a, 15b are brought together, the jaws 40a, 40b close. When the proximal ends 25a, 25b of the handles 15a, 15b are separated, the jaws 40a, 40b open.

A disposable cartridge 55 is detachably mounted adjacent to the distal end 50a of the first jaw 40a. Adjacent to the distal end 50b of the second jaw 40b is an anvil 60. The anvil 60 cooperates with the cartridge 55 and first jaw 40a to clamp layers of body tissue and to connect these tissues together with a row of staples. The anvil 60 and cartridge 55 may be any type known to one skilled-in-the-art, including but not limited to those described in U.S. Pat. Nos. 6,315,185 and 6,131,790, both issued to H. A. Piraka, and both of which are hereby incorporated by reference in their entirety. To assist in retaining the cartridge 55 on the jaw 40a, the cartridge 55 and jaw 40a may include features that releasably engage each other. For example, a small depression or recess may be formed in the jaw 40a and a correspondingly sized protrusion formed on the cartridge 55. The protrusion is received in the depression and frictionally retained there due to the sizing of the protrusion relative to the depression. As such, the protrusion and recess lightly hold the cartridge 55 in place on the jaw 40a, preventing sliding of the cartridge 55 along the jaw 40a or side-to-side swaying of the cartridge 55. Since the purpose of the protrusion and recess are to prevent sliding, the sizing of the protrusion relative to the recess is also made such that the cartridge 55 can be anchored to, and removed from, the jaw 40a with a minimal amount of manual force.

The stapling and cutting system 10 exhibits several important advantages over other know stapling and cutting devices. For example the reduced complexity of the current design 10 can (a) lower manufacturing costs; (b) allow one to readily clean and sterilize the system through the use of autoclaves and methods that are routinely utilized to clean and sterilize other surgical instruments; and (c) generate the force necessary to cut the layers of body tissue and/or fire all of the staples simultaneously through the body tissue layers and to clinch the end portions of the staples.

With the present construction, the anvil 60 and cartridge 55 are positioned such that when the jaws 40a, 40b are open, the anvil 60 and cartridge 55 are substantially parallel to and opposed from one another, and when the jaws 40a, 40b are closed, the anvil 60 and cartridge 55 maintain their substantially parallel and opposed orientation in order to engage the tissue layers and to cut and/or deliver the staples simultaneously. The ability of the jaws 40a, 40b to remain parallel and opposed to one another in both the open and closed positions allows the system 10 to accommodate the stapling and cutting of tissue having differing thicknesses.

Still referring to FIG. 1, the proximal end 25b of the second handle 15b may include a prong 70 formed therewith that is capable of mating with a latch 65 coupled to the proximal end 25a of the first handle 15a. When the operator causes the latch 65 to contact and mate with the prong 70, the stapling and cutting system 10 is maintained in its closed position. Separation of the latch 65 and the prong 70 is required to allow the operator to move the stapling and cutting system 10 to its open position. One skilled in the art will understand that the prong 70 may be located on the first handle 15a and the latch 65 located on the second handle 15b or that other means may be utilized to secure and retain the system 10 in its closed position.

The surface of one of the handles 15a, 15b (shown as the first handle 15a in FIG. 1) is formed with a recessed portion 35 between the handle's proximate ends 25a, 25b and distal ends 30a, 30b. In this recessed portion 35, the second handle 15b crosses over the first handle 15a and is pivotally coupled thereto at the pivot point 20. As such, the proximate ends 25a, 25b and distal ends 30a, 30b are substantially in plane with one another. This in turn allows the jaws 40a, 40b to be mounted to the handles 15a, 15b in plane with one another and on a common side of the handles 15a, 15b. Thus, when the stapling and cutting system 10 is in its closed position, the first and second handles 15a, 15b cause the anvil 60 and cartridge 55 to be located directly opposite of one another instead of being off-set from one another.

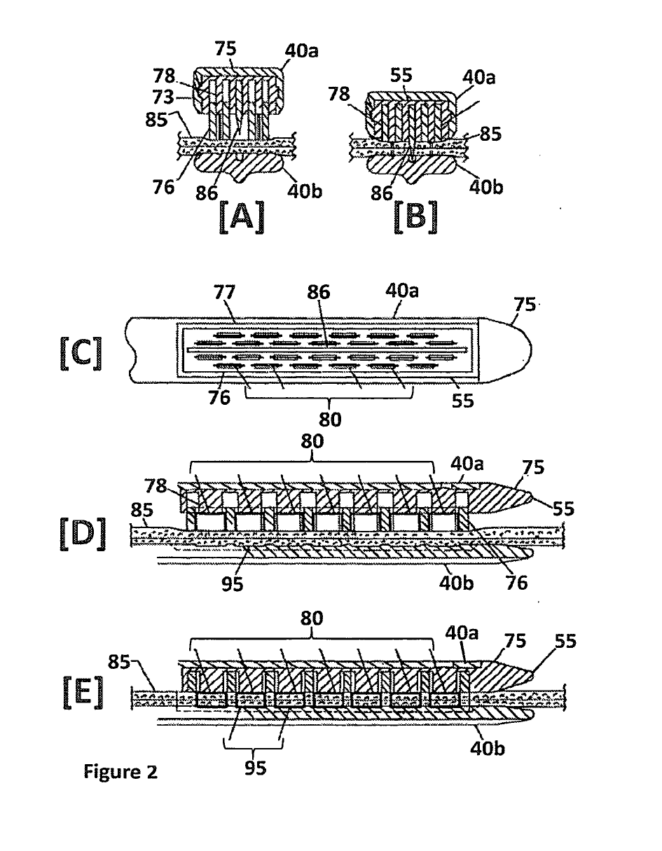

Referring now to FIGS. 2A-2E, the preferred construction of the cartridge 55 includes an upper generally rectangular shaped staple unloading member 75 and a generally rectangular shaped staple retaining member 76. The staple unloading member 75 includes an outer wall 77 and at least one pusher support 78 inside of the outer wall 77 which projects away from the outer wall 77. When the operator (not shown) forces the proximal end 25a, 25b of the handles 15a, 15b towards one another, the pusher support 78 pushes against the staples 80 to simultaneously eject and fire the staples 80 through the layers of tissue 85 and to clinch the end portions of the staples 80 as shown in FIG. 2B. Mounted in the center of the unloading member 75 is a sharp knife blade 86 for separating the tissues 85 between inner rows of staples 80 during the clinching of the staples 80.

Referring now to FIGS. 2C-2E, the staple retaining member 76 may be pre-assembled to the staple unloading member 75 by partially engaging the lower portion of the unloading member 75 with an upper portion of the retaining member 76. During a firing and clinching of the staples 80, the overall size of the cartridge 55 is reduced as the staple unloading member 75 fully engages the staple retaining member 76. The reduction in cartridge size facilitates a withdrawal of the cartridge 36 during surgery.

Referring now to FIG. 2C, staples 80 of adjacent rows are preferably staggered. Although four rows of staples 80 and a knife blade 86 are shown, it will be apparent that fewer or greater numbers of staple 80 rows can be provided, with or without the knife blade. For enhancing the readability of the figure, only a representative number of the staples 80 are identified with reference numerals.

Referring now to FIGS. 2D and 2E, the inclusion of several optional features associated with the staples 80 can effectively reduce the force requirements for firing and clinching the staples 80. These optional features include the pre-forming the hinge points associated with the staples 80 and/or using staples having sharp end portions. During the clinching of the staples 80, their pointed end portions may contact depressions 95 in the anvil 60 of the second jaw 40b, which will bend the pointed end portions of the staples 80 inwardly thereby closing/clinching the staples 80.

The detachable cartridge 55 may be bent/angled near the distal end 50A of the first jaw 40A. The lower portion of the detachable cartridge 55, which is collapsible upon the application of the force necessary to staple and cut the layers of tissue, may include a bend/angle that is sharper than the bend/angle near the top portion of the detachable cartridge 55.

Referring now to FIGS. 2A and 2B, the detachable cartridge 55 may include staples 80 in contact with a staple unloading member 75 and a staple retaining member or holder 76. The detachable cartridge may be in an extended state as shown in FIG. 2A or a collapsed state shown in FIG. 2B. Movement from the extended state to the collapsed state is a result of the force generated upon the closing of the jaws 40A, 40B and the firing of the staples 80. According to one aspect of the present invention, the staple retaining member 76 may move relative to the staple unloading member 75, thereby, moving from the extended state to the collapsed state causing the staples to be fired. In the collapsed state, the cutting blade 86 (if present) is extended to cut the tissue layers.

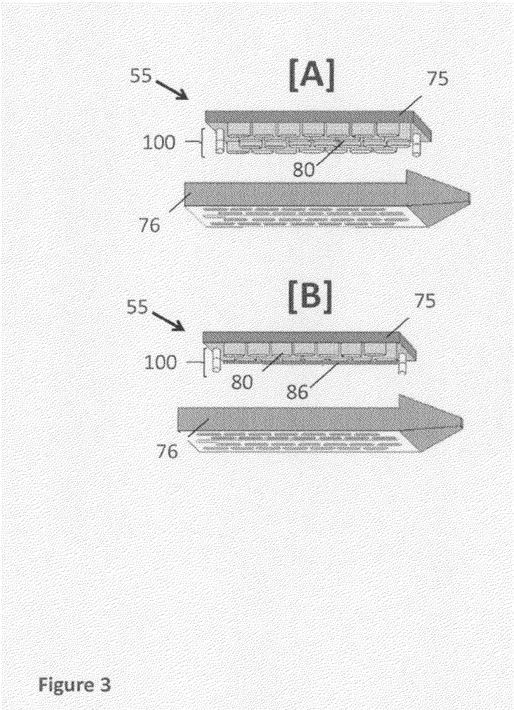

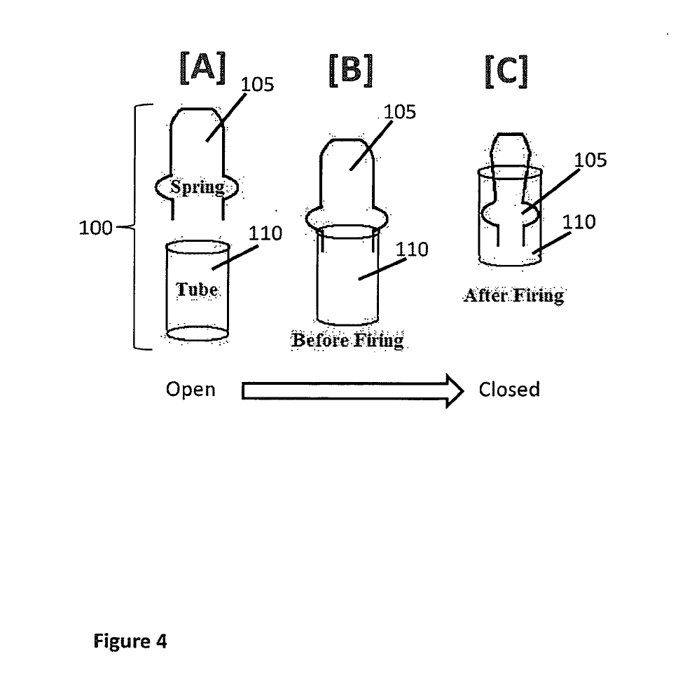

The detachable cartridge 55 typically will include at least one spring 105 and may optionally include at least one premature firing preventer 100 as shown in FIGS. 3A and 3B and 4A-4C. Preferably, the detachable cartridge 55 will include two springs 105 and/or premature firing preventers 100. The premature firing preventer 100 includes the support spring 105 and a tubular projection 110 that are in contact with one another and each of which contact with one surface of either the staple unloading member 75 or the staple retaining member 76. One purpose of the springs 105 is to assist in returning the detachable cartridge 55 to its extended state after the firing of the staples 80. In addition, the springs 105 hide the blade 86 after firing the staples 80, thereby, avoiding injury to the layers of tissue 85 by the sharp edge of the blade 86.

When the stapling and cutting system 10 is in its open position, the spring 105 and tubular projection 110 in each premature firing preventer 100 are separated (FIG. 4A). After the operator positions the tissue 85 between the jaws 40a, 40b and begins to squeeze the handles 15a, 15b together, the spring 105 and the tubular projection 110 contact one another creating a resistance to further closing of the jaws 15a, 15b (FIG. 4B). The occurrence of this resistance prevents the premature firing of the staples 80. In other words, the occurrence of this resistance provides the operator with an opportunity to reposition the tissue 85 and/or the stapling and cutting system 10 prior to applying additional force to the proximal end 25a, 25b of the handles 15a, 15b in order to fire the staples 80. This additional application of force causes the spring 105 to be received into the tubular projection 110, thereby allowing the jaws 40a, 40b to close and the stapling and/or cutting to occur (FIG. 4C).

The stapling and cutting system 10 acts as a force multiplier in that the force exerted by the physician to move the handles 15a, 15b together will be less than the force exerted by the jaws 40a, 40b when closed in order to clamp and cut the tissue layers, as well as fire and clinch the staples. One skilled-in-the-art will understand that the location of the pivot point 20 and the length of the handles 15a, 15b and jaws 40a, 40b represent design variables that can be used to adjust the overall force applied via the jaws 40a, 40b.

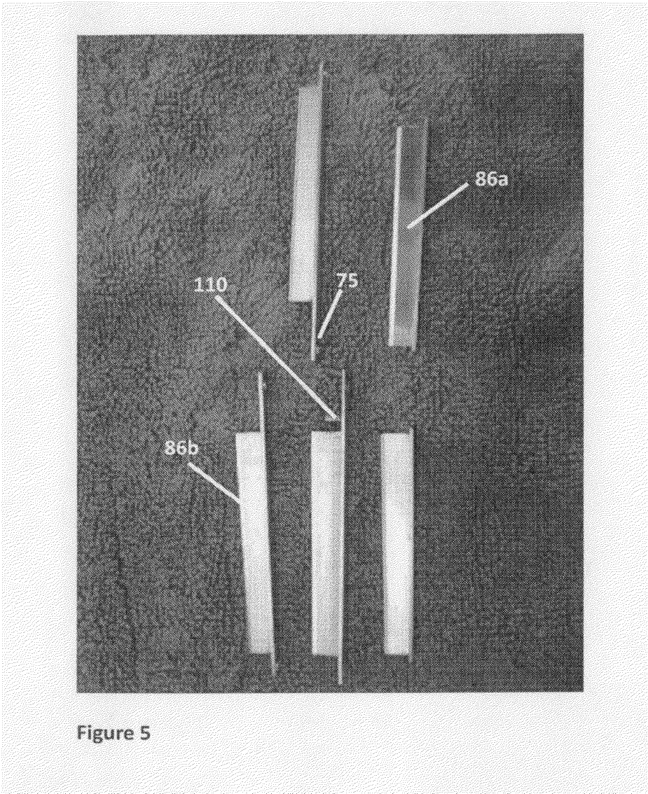

Referring now to FIG. 5, various forms of the cutting blade 86 are shown. The cutting blade 86 may have a cutting surface 86a that is entirely parallel with the staple unloading member 75. In this case, the entire cutting surface will make contact with the tissue at approximately the same time, thereby causing a chopping motion through the tissue. In another aspect of the present disclosure, the cutting blade 86 may have a cutting surface 86b that is slightly angled in order to allow the surface of the blade to contact the tissue over a given time interval, thereby, causing a slicing motion. The angle of the blade may be any angle desired by the operator, including, but not limited to about 5 degrees.

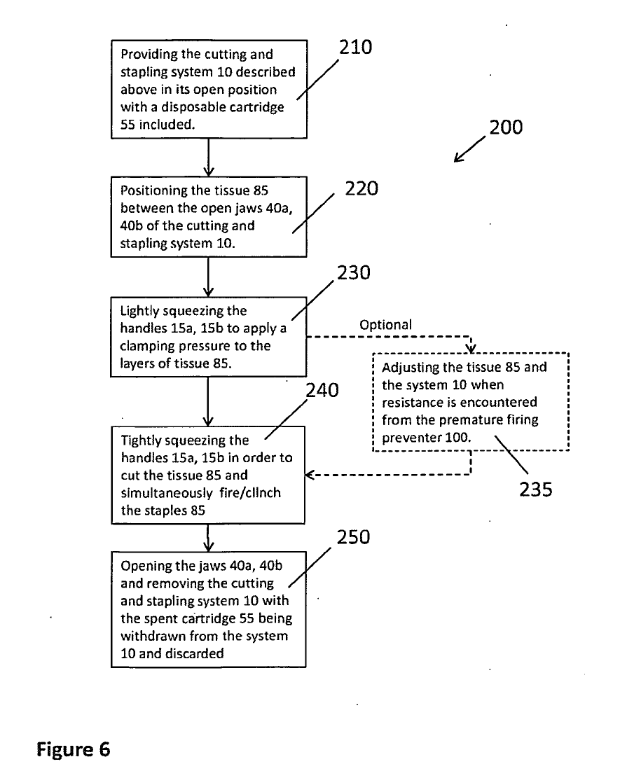

Another objective of the present disclosure is to provide a method of using the stapling and cutting system 10 described above. The method 200, set out in the flow chart of FIG. 6, generally comprises providing 210 the stapling and cutting system 10 (described above) including a disposable cartridge 55 in its open position. The operator then positions 220 the tissue 85 between the open jaws 40a, 40b of the stapling and cutting system 10. The handles 15a, 15b are then lightly squeezed 230 by the operator to apply a clamping pressure to the layers of tissue 85 in order to be sure about the exact location of the clamped tissue desired for stapling. If the operator is satisfied with the position of the jaws 15a, 15b and tissue 85, the handles 15a, 15b are next tightly squeezed at step 240 to cut the tissue and fire/clinch the staples 85. After the clinching of the staples 85, the jaws 40a, 40b are opened in step 250 and the stapling and cutting system 10 is removed with the spent cartridge 55 being withdrawn from the stapling system 10 and discarded.

The foregoing description of various embodiments of the invention has been presented for purposes of illustration and description. It is not intended to be exhaustive or to limit the invention to the precise embodiments disclosed. Numerous modifications or variations are possible in light of the above teachings. The embodiments discussed were chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

What is claimed is:

1. A stapling and cutting system for use by a physician in the cutting and stapling together of layers of body tissue, the stapling and cutting system comprising:
a first handle and a second handle, the first and second handles being pivotally coupled together at a pivot joint located between proximate and distal ends of each of the first and second handles;
a first jaw and a second jaw, the first and second jaws being coupled together and movable between an open position and a closed position;
a disposable staple cartridge mounted on a distal end of the first jaw and for positioning on one side of the layers of tissue, the staple cartridge including a plurality of staples;
an anvil mounted on the distal end of the second jaw and for positioning on an opposing side of the layers of tissue; and
a closure mechanism coupling the first and second handles to the first and second jaws and causing movement of the first and second jaws from the open position to the closed position upon manipulation of the first and second handles, the closure mechanism positioning the anvil and the staple cartridge substantially parallel to and opposite from one another when the first and second jaws are in the open position; the closure mechanism also positioning the anvil and the staple cartridge substantially parallel to and opposite from one another when the first and second jaws are in the closed position; and
whereby the staples are discharged from the system and clinched when the first and second jaws are moved from the open position into the closed position.

2. The stapling and cutting system of claim 1, wherein the closure mechanism includes first and second slots respectively defined in the first and second jaws, portions adjacent to the distal ends of the first and second handles being slidably received in and engaged with the first and second slots.

3. The stapling and cutting system of claim 2, wherein the first and second jaws have proximate and distal ends, the first and second handles being engaged in the slots toward an end of the slots located toward the distal ends of the first and second jaws when the first and second jaws are in the closed position.

4. The stapling and cutting system of claim 2, wherein the first and second jaws have proximate and distal ends, the first and second handles being engaged in the slots toward an end of the slots located toward the proximate ends of the first and second jaws when the first and second jaws are in the open position.

5. The stapling and cutting system of claim 1, wherein the closure mechanism includes a proximal end of the first jaw engaging the first handle and a proximal end of the second jaw engaging the second handle.

6. The stapling and cutting system of claim 5, wherein the proximal ends of the first and second jaws are pivotally engaged with the first and second handles, respectively.

7. The stapling and cutting system of claim 5, wherein the proximal ends of the first and second jaws are pivotally engaged with the first and second handles toward the proximate ends of the first and second handles.

8. The stapling and cutting system of claim 1, wherein one of the first and second handles includes a recessed portion located between the proximate and distal ends of the one of the first and second handles, the pivot joint being formed in the recessed portion.

9. The stapling and cutting system of claim 8, wherein the other of the first and second handles is generally planar in shape.

10. The stapling and cutting system of claim 9 wherein the first and second jaws are generally planar in shape.

11. The stapling and cutting system of claim 1, wherein the disposable cartridge further comprises a premature firing preventer, the firing preventer engaging the anvil prior to the first and second jaws being positioned in the closed position.

12. The stapling and cutting system of claim 11, wherein the firing preventer includes a biasing member that increases the force required to move the first and second jaws into the closed position relative to the force required to move the first and second jaws from the open position until the firing preventer engages the anvil.

13. The stapling and cutting system of claim 1, further comprising a blade having a cutting edge and being mounted to the staple cartridge, the blade positioned so as to pass into a slot formed in a surface of the anvil.

14. The stapling and the cutting system of claim 13, wherein the cutting edge is parallel with respect to the surface of the anvil.

15. The stapling and the cutting system of claim 13, wherein the cutting edge is angled with respect to the surface of the anvil.

16. A method for discharging a staple from a stapling and cutting system, the method comprising the steps of:
providing a stapling and cutting system, the stapling and cutting system having a first jaw and a second jaw; the jaws being movable between open and closed positions and being operable by manipulation of a first handle and a second handle, a disposable staple cartridge located on the first jaw and including multiple staples, an anvil located on the second jaw generally opposite from the staple cartridge, a closure mechanism that couples the first and second handle to the first and second jaws;
moving the first and second handles toward each other under a first pressure to move the jaws from the open position toward the closed position while maintaining the staple cartridge and the anvil generally parallel to each other;
while keeping the staple cartridge and anvil substantially parallel to each other, moving the first and second handles toward each other under a second pressure and causing the jaws to move into the closed position, the second pressure being greater than the first pressure;
squeezing the handles at the second pressure whereby at least one staple is discharged from staple cartridge and clinched by interaction with the anvil; and
opening the jaws to permit removal discarding of the staple cartridge from the system.

17. The method of claim 16, further comprising the step of providing increased resistance whereby the applying of the first pressure is insufficient to move the first and second jaws into the closed position.

18. The method of claim 17, wherein the moving of the first and second jaws is substantially parallel to each other.

19. The method of claim 18, wherein moving of the first and second handles is not substantially parallel to each other.
